(19) United States
(12) Patent Application Publication
(10) Pub. No.: US 2016/0346566 A1
(43) Pub. Date: Dec. 1, 2016

(54) OPTIMISATION OF A METHOD FOR CALCULATING DOSES DEPOSITED BY AN IONISING BEAM

(71) Applicant: COMMISSARIAT A L'ENERGIE ATOMIQUE ET AUX ENERGIES ALTERNATIVES, Paris (FR)

(72) Inventors: David MERCIER, Dourdan (FR); Ming-Chao YANG, Massy (FR)

(21) Appl. No.: 15/110,369

(22) PCT Filed: Jan. 28, 2015
(86) PCT No.: PCT/EP2015/051715

§ 371 Date: Jul. 7, 2016

(30) Foreign Application Priority Data
Feb. 20, 2014 (FR) 1451358

(51) Int. Cl. A61N 5/10

(52) U.S. Cl. CPC A61N 5/1031; A61N 5/1083; A61N 5/1081; A61N 2005/1074; A61N 2005/1087; A61N 2005/1089

(57) ABSTRACT

A method for estimating a dose gradient of the parameters of a beam of ionizing particles, the dose being deposited by the beam in a voxel of a meshed phantom of a patient, each mesh cell comprising voxels of one and the same material, the parameters of the beam comprising a fluence parameter and geometric parameters, the method comprises the determination of the analytic function for the gradient of the dose, deposited per mesh cell, of the parameters of the beam; and the determination of the estimation of the gradient of the dose in the voxel. Certain procedures for estimating the dose deposited are described. Developments deal with configurations having several independent of partially dependent irradiating beams, optimizations of the treatment plan using cost functions and the management of beams moving along trajectories. The use of servo-controlled robotic arms or of other mobile systems during irradiation is described.

### FIELD OF THE INVENTION

The invention relates to the optimization of a method for calculating doses deposited by an ionizing beam, for example used by a radiotherapy based therapeutic treatment device. On account of recent technologies in radiotherapy (arc therapy, tomotherapy, “cyberknife”), the ionizing beam can be moving.

### PRIOR ART

By means of medical scanners, the geometry of the body of a patient, of their tissues and of possible tumors can be established. Regions of like electron density can be grouped together. The doctor then determines the regions to be irradiated and safeguarded, possibly with certain safety margins (slight movement of organs, skill of the doctor, protocols, etc) as well as the (minimal and/or maximal) associated doses.

The general technical problem then consists in optimizing dose deposition by ionizing beams in tissues in order to irradiate tumors to at least a minimum, healthy tissues to at most a maximum and in avoiding certain others if possible (for example the spinal cord).

The beams are controlled (position in space, intensity or fluence) and the establishment of a treatment plan is aimed at optimizing the distribution of these irradiation doses.

Patent EP10732960 entitled “Procédé de calcul de doses déposées par un rayonnement ionisant” [Method for calculating doses deposited by an ionizing radiation] (Blanpain, Mercier, Barthe), which method is entitled “Doséclair” in what follows, discloses, —such as stated in its abstract —, a calculation method comprising at least a first step of calculating a distribution function for the dose in mesh cells of a meshed phantom, a second step of calculating the dose deposited in a set of voxels, the value of the dose deposited for a voxel being given by the distribution function for the dose specific to the mesh cell to which the voxel belongs. The invention applies to intensity-modulated (fluence) radiotherapy.

This procedure, although efficacious, is still not fast enough to allow a procedure for exhaustive optimization of the irradiation parameters (that is to say by testing all possible solutions in order to select the best one among them) in order to comply best with the doctor's prescription.

In situations where the irradiation means are moving (for example with a servo-controlled robotic arm carrying the irradiation means or according to a system of arc therapy or tomotherapy type), the technical problem posed by the requirement for fast dose calculation remains unresolved and there are still no satisfactory answers for approaches based on intensive calculation.

### SUMMARY OF THE INVENTION

There is disclosed a method (for example implemented by computer) for estimating a dose gradient with respect to the parameters of a beam of ionizing particles, the dose being deposited by the beam in a voxel of a meshed phantom of a patient, each mesh cell comprising voxels of one and the same material, the parameters of the beam comprising a fluence parameter and geometric parameters, the method comprising the determination of the analytic function for the gradient of the dose, deposited per mesh cell, with respect to the parameters of the beam; and the determination of the estimation of the gradient of the dose in the voxel. Certain procedures for estimating the dose deposited are described. Developments deal with configurations having several irradiating beams, be they independent or partially dependent, optimizations of the treatment plan using cost functions and the management of beams moving along trajectories. The use of one or more servo-controlled robotic arms or of helical or mobile systems is described.

The method as a whole, and/or each and/or some of the steps of the method can be implemented by computer, e.g. calculation means (for example a processor executing instructions) can allow the implementation of any one of the steps of the method.

Numerous advantages ensue from the invention.

No procedure for calculating dose has existed hitherto which enabled the gradient of the dose with respect to the geometric parameters of the beams to be made explicit. Various described aspects of the method make it possible in particular to optimize all the parameters simultaneously. The method makes it possible in fact to simultaneously calculate the gradient on the fluences and, at the same time, the gradient of the dose with respect to the geometric parameters (something which no prior art document mentions). This entails a major concrete advantage. A new configuration of the beams and of the associated fluences can be optimized, all the parameters being taken into account simultaneously. Stated otherwise, the beams according to the invention are “determined” simultaneously: the process of irradiation according to one beam can be adapted as a function of what the other beams are doing. Multiple optimizations then become possible (irradiation by moving beams, dilution effects and better distribution of the doses deposited, etc).

The calculation speed permitted by the proposed method makes it possible to derive the analytic formulation, for example provided by “Doséclair”, and to apply an iterative optimization process, for example by gradient descent, which involves the whole set of parameters of the beam. A cost function being defined to qualify the relevance of a planned treatment in relation to objectives of minimal or maximal dose in the various organs, the proposed method makes it possible to give an analytic formulation of the gradient of this cost function in the mesh cells, still with respect to the parameters of the beams.

An advantage associated with the disclosed method also lies in the fact that the calculations can be fast. The calculation codes can be optimized and parallelized (each elementary beam and then each voxel can be evaluated independently of the others, doing so in correspondence with the multi-core architectures of the processors). The corresponding calculation speed makes it possible to propose a treatment plan rapidly (typically less than 20 minutes, e.g. the time required for a consultation and discussion about the principle of the treatment), in order for example that the appointment can be made immediately at the conclusion of the consultation.

### DESCRIPTION OF THE FIGURES

Various aspects and advantages of the invention will be apparent in support of the description of a preferred but nonlimiting mode of implementation of the invention, with reference to the figures hereinbelow:

FIG. 1 illustrates by examples the notion of “phantom” of a patient, of “mesh cell” of a phantom and of “voxel” of a mesh cell;

FIG. 2 illustrates an exemplary advantageous system of coordinates for defining the geometric parameters of an ionizing beam;

FIGS. 3A and 3B illustrate various examples of embodiments of the invention, the beam of ionizing particles being in motion;

FIG. 4 schematically illustrates steps of the method, especially for estimating the dose gradient.

### DETAILED DESCRIPTION OF THE INVENTION

Reference is made to the content of patent application EP10732960 published on 20 Jan. 2011 (WO2011/006907A1), therefore accessible to the public, entitled “Procédé de calcul de doses déposées par un rayonnement ionisant”[Method for calculating doses deposited by an ionizing radiation]” (Blanpain, Mercier, Barthe). Reference will be made to this publication under the name “Doséclair”.

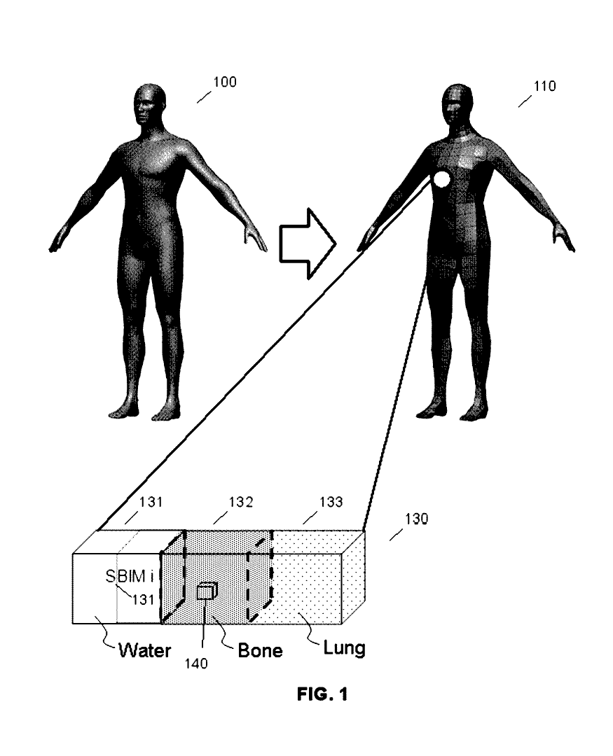

The definitions and interpretation to be afforded to the terms of the present patent application do not stray from this initial application, nonetheless certain definitions are recalled hereinafter in respect of FIG. 1.

FIG. 1 illustrates the notions of phantom, voxel and mesh cell. The phantom of a patient 110 is a three-dimensional matrix representation of a part of the body of the patient 100.

In a phantom 110, composed of mesh cells 130 (for example a mesh cell of water 131, a mesh cell of bone 132 or of lung 133), each voxel (for example 140) is characterized by the fact that it consists of one and the same material and/or one and the same electron density.

A voxel (for example 140) is to a volume what a pixel is to a surface (image). The term voxel should be interpreted in a generic sense, in its broadest acceptance. The voxel is the “atomic” unit. The voxel generally corresponds to the unit of resolution of the equipment. A tumor may be of the same density as the surrounding tissues. The drop to voxel level is necessary in order to see anatomical elements that cannot be distinguished on the scale of a mesh cell.

A mesh cell (131, 132, 133) corresponds to a cluster of adjacent voxels. The mesh may be regular or irregular, the mesh cells possibly being of different dimensions.

The dose deposited is calculated with the aid of an analytic form. For example an analytic form can correspond to the composition of a projection function and of a precalculated dose distribution function. By multiplying this normalized dose distribution by the initial fluence of an elementary beam, an associated dose distribution is obtained.

A single analytic function is associated with the set of voxels of one and the same mesh cell. Stated otherwise, to know which analytic function is associated with a given mesh cell, the voxel of interest is examined. Said voxel is associated with a certain position inside a certain mesh cell of the phantom. The same analytic function is used for several voxels of one and the same mesh cell. The evaluation of the analytic function may differ from voxel to voxel. Two given voxels may correspond to two different mesh cells. The evaluation of the analytic function may also differ from voxel to voxel, even within the same mesh cell, since the position is an input variable of the analytic function (this is the P in the equations).

The analytic functions according to the invention are concretely implemented in the form of algorithms (or of a collection of algorithms). There is a great diversity of algorithms that can be used to implement one and the same (mathematical) analytic function. There is also a variety of analytic functions that can lend themselves to the various embodiments of the invention. These functions can therefore take different forms and/or be obtained by different pathways from those presently disclosed (“Doséclair”). The analytic functions according to the invention correspond to continuous, differentiable and additive functions.

A gradient is an operator which quantifies to first order the variation of a function induced by the variations of the variables on which it depends.

An SBIM (Sub-Beam In Mesh) 131 appearing in FIG. 1 and detailed in “Doséclair” represents a sub-part of the elementary beam in a mesh cell, corresponding to all the photons which have a primary interaction with the matter in a particular volume of the phantom so that: the medium of this volume is homogeneous; the fluence over a section is constant or varies very little. If necessary therefore, the beam can be cut up into SBIMs that are sufficiently narrow for the variations of their fluence not to be significant over the section. The use of SBIMs must be seen as a convenience of calculation and is not in any case an essential characteristic. Reference is made to the “Doséclair” patent.

The dose calculation method is not directly derivable, at least not analytically. In fact a parameter exists which cannot be treated analytically and it is this parameter whose derivative is calculated by propagation. This parameter conveys the heterogeneities of the medium, including the “void” region between the beam origin and the body of the patient. Axial propagation consists in calculating gradually, in a recursive manner, the dose deposited. Lateral propagation corresponds to the fact that a change of material in the neighboring mesh cell may have consequences for the lateral decay of the dose. Finally, multiple lateral propagation is also conceivable with the aid of the principle of mesh cell to mesh cell propagation. The tensorial calculation associated with the propagation step according to the invention integrates the whole set of these propagation modes.

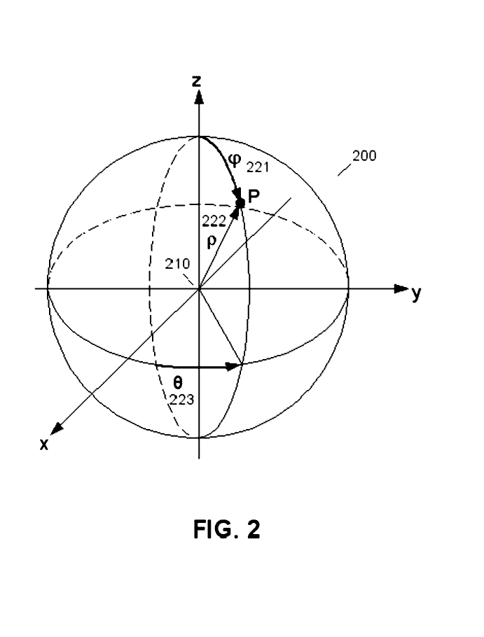

FIG. 2 illustrates the degrees of freedom of a beam in a particular embodiment. A beam of ionizing particles (“irradiating beam” or “irradiation beam”) is characterized by its direction in space and its fluence (more precisely the spatial modulation in fluence corresponds to the intensity of the beam). FIG. 2 illustrates a particular and optional system of coordinates which lends itself to the tensorial calculation for determining the gradients and the operations for propagating the analytical parameters in a particularly advantageous manner. According to this system of coordinates, a beam exhibits five degrees of freedom, corresponding to the information regarding the origin, rotation and direction of the beam in space: the origin P of the beam is determined on the basis of the origin center of the phantom 210 via the angles θ (longitude, 223) and φ (colatitude, 221) and R the radius of the sphere which is considered to be constant here (and therefore not to be a parameter). The orientation of the beam is determined by the triplet (α; β; γ) according to the Euler angles (here the variant named Tait-Bryan z-y-x or the succession of rotations is defined by a rotation by α about the axis z, a rotation by β about the new axis y (sometimes also denoted y′), a rotation by γ about the new axis x (sometimes also denoted x″).

The beam M is entirely determined by the quintuplet (θ; φ; α; β; γ). According to this system of coordinates, it is possible to demonstrate by tensorial calculation operations that it is possible to obtain the gradient of the dose deposited at each point of the phantom with respect to the parameters of the beam. It is stressed that this frame of reference as well as the associated mathematical notation are optional, in the sense that it would not entail an essential characteristic but merely a convenience of calculation (by compensation in the calculation of the tensors).

By writing for example Ψ_{f}=(θ_{f}; φ_{f}; α_{f}; β_{f}; γ_{f}) for the unitary elementary beam parameters and G_{α} for the gradient of the tensor with respect to α_{f}, (gradient tensor α), the formulae that can be derived between the coefficients of the tensor and the parameters make it possible to calculate the gradient at any point, with the exception of a coefficient t₃₄ (see Annex) which can only be calculated by propagation (gradual calculation). Thus, each gradient tensor can be defined analytically. It is possible to calculate the gradients at the points of interest alone. It is possible to obtain the gradient of the dose deposited at each point of the phantom with respect to the parameters of the elementary beam.

The method as such operates in the manner described hereinafter.

There is disclosed a method for estimating a dose gradient with respect to the parameters of a beam of ionizing particles, the dose being deposited by said beam in a voxel of a phantom of a patient, said phantom being meshed, each mesh cell of the phantom comprising voxels of one and the same material, the parameters of the beam comprising a fluence parameter and geometric parameters, said method comprising the determination of the analytic function for the gradient of the dose, deposited per mesh cell, with respect to the parameters of the beam; and the determination of the estimation of the gradient of the dose in the voxel.

It is recalled that the term “voxel” should be interpreted, if need be, in a generic sense (for example by analogy with the “pixel” of an image). The determination of the analytic function for the gradient of the dose deposited per mesh cell is performed with respect to the parameters of the beam, which parameters in fact include both the fluence parameter of the beam and the geometric parameters of the beam. The term “estimation” may (in particular) be interpreted as a “calculation” (rough or approximate). The “real” value of the gradient exists but this value is not directly accessible, i.e. no formula for calculating it directly and exactly is made formally explicit (its most precise possible approximation is precisely the subject of the present disclosure). Under these provisos, the term “calculation” can be substituted for the term “estimation”.

In a development, the method of “Doséclair” is invoked to estimate or determine the dose deposited. This combination remains optional, other procedures for calculating dose deposited being possible. According to this embodiment, there is disclosed a method for estimating a dose gradient, the estimation of the dose gradient being the gradient of the dose estimation, and for which the estimation of the dose deposited in the voxel comprises the determination of an analytic function for calculating dose deposited per mesh cell, said function being obtained by propagating the parameters of analytic functions of dose deposited gradually in the neighboring mesh cells traversed by the beam; and the determination of the estimation of the dose deposited in the voxel.

The estimation of the dose gradient is the gradient of the dose estimation. The known “Doséclair” method discloses a procedure for estimating dose (for calculating dose). This particular “Doséclair” method for estimating dose can be summarized or interpreted as follows: “estimation of the dose deposited in the voxel [comprising] the determination of an analytic function for calculating dose deposited per mesh cell, said function being obtained by propagating the parameters of analytic functions of dose deposited gradually in the neighboring mesh cells traversed by the beam; and the determination of the estimation of the dose deposited in the voxel”. This estimation procedure is advantageous but is not the only possible one. Stated otherwise, this calculation method remains entirely optional, that is to say discretionary. Again stated otherwise, it would not be necessary, or legitimate, to constrain to a restriction or a limitation consisting in integrating this estimation procedure into the method for estimating the dose gradient such as presently disclosed. Other procedures for estimating dose include—but are not limited—to algorithmic formulations (that is to say which are not necessarily in the form of functions; stated otherwise, running a program in the course of time may allow the expression of an estimation of the dose and this program may not reduce to a mathematical function). Charts or other heuristics or indeed direct measurement procedures also remain possible.

In a development, the method can provide for the use of a plurality of independent beams. In this case, the method can comprise, furthermore, the determination of the dose deposited in the voxel by summing the doses deposited by each independent beam.

In the case of a plurality of beams, the beams may be dependent or independent. Specifically for example, the displacement of a beam may or may not influence the displacement of other beams. The doses deposited are additive. Depending on whether the beams are or are not independent (i.e. at least partially dependent), the determination of the total dose deposited can be performed in various ways. It is for example possible to determine the global dose by summing the doses deposited by each beam (additivity). The global or total gradient is obtained by summing the gradients of each beam.

In the case of independent beams, the (geometric) parameters associated with the beams are independent, that is to say that no mathematical relation exists between them. Stated otherwise, independent beams do not have any interdependent geometric parameters. Knowing certain parameters of certain beams does not make it possible to deduce the others therefrom. In the case of dependent beams, the parameters are dependent, at least in part, that is to say that a relation exists between them. For example, if the centers of four beams form a square, then knowing the first three centers enables the determination of the fourth center at the fourth vertex of the square). If there is independence, such a deduction is impossible. In the case of “Cyberknife” (described hereinafter) or of so-called “step and shout” beams, there may be independence.

In a development, still in the case of a plurality of independent beams, the method can comprise the estimation of the gradient of dose deposited in the voxel by summing the gradients of doses deposited by each independent beam. The global or total gradient is obtained by summing the gradients of each beam. Specifically, this addition is performed on gradients that may be regarded as “extended”, i.e. in a mathematical notation format which makes the summation possible. Simplifying, for each (individual) beam, the gradient of the beam with respect to the parameters of the beam is padded with Os at the appropriate locations to obtain the gradient with respect to the union of the parameters of all the beams. This operation being repeated for each beam (the zero values are therefore not at the same locations), the gradients are therefore summable and are in fact summed.

In a development, this time in the case of a plurality of at least partially dependent beams, some of their geometric parameters being interdependent, the method can comprise, furthermore, a step of determining the dose deposited in the voxel. In the case where the beams are not independent but at least partially dependent (mathematical relations exist between certain of the parameters of at least some of the beams, e.g. some of their geometric parameters being interdependent), the gradient is then evaluated with respect to a set of parameters selected from the union of the parameters of the various beams so as to all be independent and/or of meta-parameters from which are deduced certain parameters of several beams, by summing the “extended” gradients of each beam.

Several types of dependency of the beams exist. In IMRT systems, the “main” beam is said to be the sum of the small adjacent beams. The dependency is therefore two-fold. All the directions of the beamlets constituting the “main” beam are identical (the alphas, betas, gammas). The centers of the beam lets are distributed over a regular grid (generalization of the simplified example of the square described above). The dependency is partial for IMRT since the fluences are actually independent and the presence of three independent main IMRT beams gives rise to three sub-groups of beam lets which are dependent in the sub-group, but each sub-group is independent of the other sub-groups.

The configuration of continuous irradiation along a trajectory is particular. Several particular modes are described: arc therapy, tomography and “cyberknife”. Arc therapy is a dynamic approach which varies both the position of the leaves and the arm angle of the apparatus during irradiation. An arc is defined by two extreme positions between which the beam is present throughout irradiation. The complete arc is approximated by a series of uniformly spaced fixed fields called control points. The intensity is optimized for each control point. Tomotherapy takes the form of an annulus that rotates around the treatment table on which the patient is installed, said annulus being composed of a linear accelerator with a binary collimator. Each complete rotation of the linear accelerator can be approximated by a series of discrete steps. Tomotherapy (through the nature of the trajectory and the binary collimator) can be considered to be a very particular case of arc therapy. Cyberknife is a device tying a linear accelerator producing a fine beam to a robotic arm, said arm making it possible to move the linear accelerator freely around the patient, thus being able to produce almost any trajectory for the fine beam.

In this case, there is “dependency” via the trajectory. It is then possible (but optional) to vary the fluences along the irradiation trajectories (for example progressively). If appropriate, there would also be dependencies on the beam fluence parameter. For example, by fixing an initial beam with a fluence F1, a final beam with a fluence F2, and an interpolation between the two, a mean beam with a fluence of (F1+F2)/2 will exist at a given moment on the trajectory (between the start and finish positions and in an intermediate direction between the start direction and the finish direction).

In a development, the determination of an analytic function for calculating gradient of dose deposited in an arbitrary voxel of the phantom is obtained through the gradient of the composition of the projection function with the function of the dose deposited in a voxel; the determination of the analytic function for calculating dose gradient being calculated gradually in the mesh cells traversed by the beam.

Stated otherwise, the analytic function for estimating dose deposited for a mesh cell is the composition of a dose distribution F with a projection function G, giving for a voxel centered at the point P a dose evaluated by F·G(P)=F(G(P)), the analytic function for calculating dose gradient being determined gradually in the mesh cells traversed by the beam by ∇(F·G)=∇G. (∇F·G), i.e. a gradient at the point P which is equal to ∇_{P}(F∇G)=∇_{P}G.∇_{G(P)}F.

For multi-variable functions, this development conveys the formula (better known) for the derivative of the composition of two single-variable functions.

This development returns to the various possible procedures for estimating dose, of which the aforementioned “Doséclair” procedure is one example among others. In particular, according to the latter model, it is possible to resort to the notion of “heterogeneous” phantom and of “homogeneous” phantom which is associated with precalculated dose distributions. If appropriate, the projection function G operates between the heterogeneous phantom of the patient and a homogeneous phantom associated with precalculated dose distributions.

In a development, it is specified that the geometric parameters of a beam can comprise five degrees of freedom, corresponding for example to the information regarding origin, rotation (i.e. two degrees) and direction of the beam in space (i.e. three degrees). Other coordinate systems are possible. The coordinate system presented lends itself well to the calculations.

In a development, there is disclosed a method (of optimization) comprising the steps of selecting a differentiable cost function; the determination of the gradient of said cost function with respect to the parameters of the beam, said gradient being obtained by composition of the derivative of the cost function with respect to the dose with the gradient of the dose with respect to the parameters; and the minimization of said cost function corresponding to the obtaining of a local minimum of the cost function. According to this development, the method can indeed be aimed at optimization (in particular of the treatment plan). Accordingly, a differentiable (or at least weakly differentiable) cost function can be used.

In a development, the method comprises the determination and the minimization of the cost function for a plurality of voxels which may or may not belong to one and the same mesh cell. It is also disclosed that the method comprises the determination and the minimization of the cost function for a plurality of voxels. The expression “which may or may not belong to one and the same mesh cell” is discretionary and constitutes an explicit reference to the fact that the irradiation can reach numerous mesh cells (overflows and overlaps for example). The irradiation may in fact “overflow” into neighboring or adjacent mesh cells. The cost function is indeed evaluated for a plurality of voxels, which is the smallest unit or subdivision used.

The treatment plan can aim to deliver minimal doses at the level of the tumor, and maximal doses elsewhere (sensitive tissues). Each voxel can have its own threshold. At the level of the tumor, a minimal dose can be imposed to guarantee that the cells of the tumor are all destroyed. Elsewhere, a maximal dose can be imposed to limit the side effects of the treatment on healthy cells, in particular the induced tumors which show up a few years later. The maximal dose is not necessarily the same everywhere, certain tissues (optic nerve, spinal cord) being much more sensitive and vital than others.

The calculation method according to the invention therefore uses also a cost function. A “cost function” is a real-valued mathematical mapping (or a function) which makes it possible to evaluate the performance of a parametrized model, according to a certain criterion. Thus, the parameters of the best model are determined by minimizing the cost function. In a configuration, for example, the parameters can be the fluences, directions and positions of the beams. In the case of a particular voxel, an example of objectives that should be conveyed by the cost function may be: a) if a minimal dose is sought, then the smaller the dose deposited relative to this dose, the bigger the cost function must be. Once exceeded, it must remain very low, or indeed zero; b) if a maximal dose is sought, then the cost function must increase rapidly if the dose deposited exceeds this threshold. At the same time, it must be low when the level delivered is smaller, but ideally zero if (and only if) no dose is deposited; c) globally, doses or errors that are very spread out are preferred. For example for a total of 1 mGy deposited on 2 voxels, preference will be given to a solution which deposits 0.5 mGy in each voxel rather than 1 mGy in one and 0 in the other. In all cases, the cost function must be differentiable (or at least weakly differentiable).

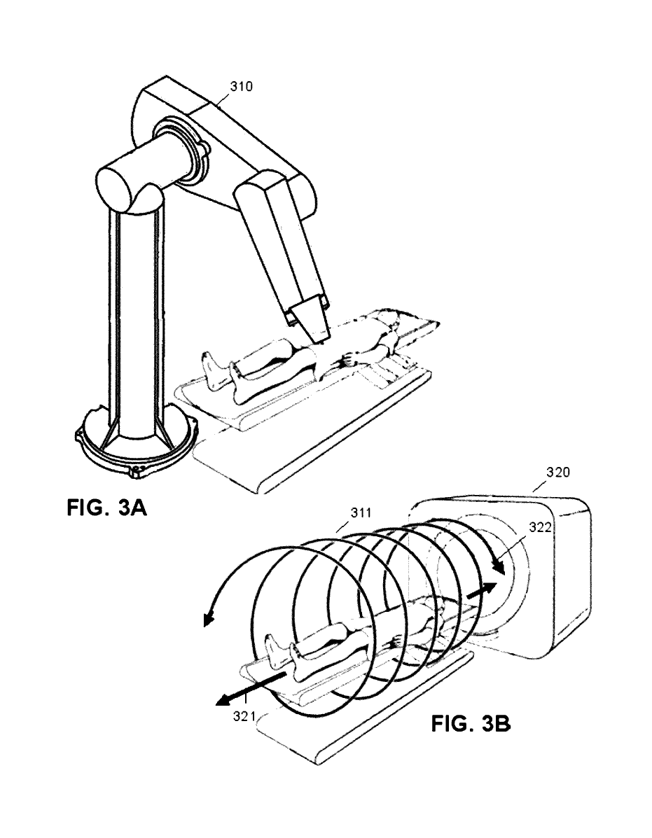

FIGS. 3A and 3B illustrate examples of embodiments of the invention, the beam of ionizing particles being in motion. FIG. 3A illustrates a robotic arm carrying one or more beams, the servo-controlled robotic arm being mobile around the patient, in a configuration called “Cyberknife”. FIG. 3B illustrates an exemplary “helical” system that combines translations and rotations. It should be noted that arc therapy covers configurations or situations that surpass helical systems such as these.

In a development, one or more beams are moving. A beam can be moving along a trajectory comprising at least two control points and intermediate trajectory points. In a development, there is disclosed a system for the implementation of any one of the steps of the method. This system can comprise a servo-controlled robotic arm carrying the beam or beams. This system, as a supplement or independently, can comprise a system of arc therapy type (irradiation beam mobile during irradiation). In a development, an optimization of the treatment plan can be based on optimizing the irradiation parameters associated with the control points. In particular, the irradiation parameters associated with the intermediate trajectory points can be deduced by interpolation of the irradiation parameters associated with the control points.

A “brute force” approach based on the known procedures, which are less sophisticated in spite of considerably enhanced calculation means, cannot in fact solve the technical problem posed by the very fast dose calculation required in mobility situations. Placed in situations in which the beams are moving continuously, the known approaches would indeed be incapable of providing satisfactory calculation solutions (prohibitive calculation time, technical difficulties related to latency time, signal transmission time, etc. . . . ).

By contrast, the developments disclosed in the present patent application make it possible to envisage “mobile” applications of this type since the calculation steps are considerably optimized. The analytic gradient calculation (or estimation) such as presently disclosed and claimed paves the way for all new applications in regard to radiotherapy, especially in regard to lightweight or even mobile solutions. It is indeed possible to undertake trajectory optimization in the case of arc therapy, tomotherapy or “Cyberknife”. Accordingly, it is possible to consider to a first approximation that the integration of the dose over the trajectory is equal to the sum of the doses with points with origin spread regularly along the trajectory. By assuming that this trajectory is defined by a small number of reference values (of an equation) or of reference points (the breaks over a broken-line trajectory), it is possible to establish the relations between these references and all the ballistic parameters of said intermediate beams. A gradient linking the dose deposited over the trajectory to these references can therefore be calculated or can be utilized to optimize all the parameters of the trajectory, with a cost in terms of calculation power that is equivalent to that for static IMRT (also termed “step&shoot”).

According to this type of configuration, it is possible to employ apparatuses that are capable of irradiating according to or with movements (of any nature, e.g. permanent or intermittent or periodic, continuous or discontinuous, etc), this having a noteworthy effect of diluting the doses for the surrounding healthy tissues. For its part, the “brute force” approach on trajectories is becoming very problematic. For example, the trajectories can be defined by broken lines. To a first approximation, the doses can then be interpolated. In the case of a linear interpolation, the dose delivered over the trajectories can be optimized. A reduced number of control points make it possible to control dose delivery. Since other interpolation procedures may be used, particular optimizations of trajectories can be calculated.

FIG. 3A illustrates a so-called “Cyberknife” configuration showing a servo-controlled robotic arm 300 carrying one or more irradiating beams 310. The arm moves around the patient. There may also be several robotic arms of this type disposed around the patient. The robotic arm can be steered in an entirely automatic or preprogrammed manner. Optionally it can also be steered by the operator by tele-guidance (in certain cases of radio-surgery). Steering can, at the very least, be assisted, i.e. partially automated. The arm can be remotely controlled if network latency times so permit (techniques exist which are aimed at reducing latency times to their strict minimum by optimizing the signal processing chains). The advantages of this configuration are to do with a very high precision as regards the placement of the ionizing beams as well as associated movements which may be very fast, reducing the session time to what is strictly necessary (that is to say to the time for the actual irradiation).

FIG. 3B shows a so-called “helical” configuration 320, combining translations 321 and rotations 322. This configuration allows practically all beam placements (in particular, irradiations directed under the body of the patient). Hospital centers are provided with such apparatuses which can be modified accordingly (adaptation of the software layers for driving the ionizing beams or irradiating heads).

The servo-controlled robotic arm may optionally comprise the appropriate instrumentation, such as one or more position and motion sensors (accelerometer, gyroscope, etc) which enable the geometric parameters and the fluence of the beam to be ascertained sufficiently precisely. In certain cases, the fluence of the beam can be adapted, for example as a function of the movements of the ionizing beams. For example, guidance in the authorized ranges of movement can (optionally) be provided to the operator (visual guidance by laser projection and/or sound projection in the event of straying outside the spatial ranges or “corridors” authorized or provided for, haptic feedbacks, etc). Augmented or virtual reality systems (e.g. headset or semi-transparent or opaque immersive glasses) can also be used as a supplement to assist the operator in the irradiation procedure or to enable operations in progress to be viewed.

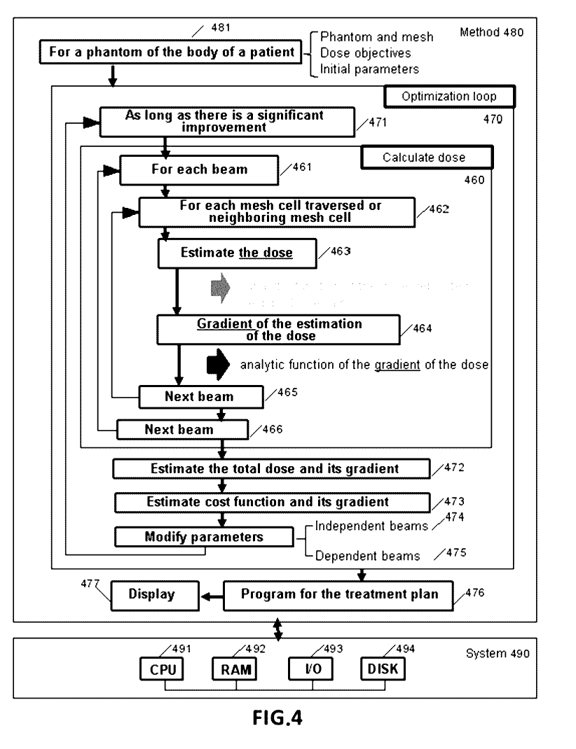

FIG. 4 illustrates certain aspects relating to the systems and to the methods according to the invention. Block 480 is a general scheme illustrating the operation of the method and its main steps. Certain steps of the method can be performed sequentially, others in parallel. The indications of the scheme are therefore illustrative and nonlimiting. Block 490 illustrates that the method can be implemented by computer.

A mesh, dose objectives (for example minimal and/or maximal dose) and initial parameters (for example in regard to geometric and/or fluence parameters of the ionizing beams) are for example defined for the phantom of the body of a patient 481. An optimization loop 470 comprises a step of calculating or estimating the function of the dose deposited 460 (by propagation through the mesh cells so as to obtain the analytic function of dose deposited). The total dose deposited by the various beams (if appropriate) as well as the gradient of the dose deposited are estimated at the evaluation step 472, for example for the voxels of interest. In step 473, by means of a cost function and of its gradient, it is then possible to determine the optimized parameters of the independent beams 474 and/or dependent beams 475. The previous steps are repeated as long as the improvements are significant (loop 471). Predefined thresholds or various other quantitative criteria can be used to establish that the improvement in the dose distribution is optimal (adjustments of the step size, etc). Subsequently, the program of the treatment plan is determined in step 476 and optionally displayed (in full or in part) in step 477.

In detail, the dose calculation 460 comprises various sub-steps. For each beam 461, for each mesh cell traversed or neighboring mesh cell 462, the analytic function of the dose deposited is calculated (or estimated) in step 466. More precisely, an analytic function of dose deposited can be determined. Various procedures exist for doing this. One way corresponds to the procedure described in “Doséclair” (but this is not the only possible procedure). Once the analytic dose function has been determined, the analytic function for the gradient of the estimation of the dose is determined in step 464. The analytic function for the gradient of the dose is obtained. Repetition continues, proceeding to the next beam 465 for each mesh cell traversed or neighboring mesh cell. When all the mesh cells considered have been processed, the method is repeated until such time as all the ionizing beams (if appropriate) are taken into account. At this instant, c.f. step 472, the total dose can be estimated or calculated (i.e. over all the mesh cells and all the beams).

There is described a method for estimating a dose gradient with respect to the parameters of a beam of ionizing particles, the dose being deposited by said beam in a voxel of a phantom of a patient, said phantom being meshed, each mesh cell of the phantom comprising voxels of one and the same material, the parameters of the beam comprising a fluence parameter and geometric parameters (comprising for example information regarding the origin, rotation and direction of the beam in space), said method comprising the determination of the analytic function for the gradient of the dose, deposited per mesh cell, with respect to the parameters of the beam; and the determination of the estimation of the gradient of the dose in the voxel. It is recalled that several procedures are possible to determine the analytic function of the dose deposited (for example, with the aid of the procedure disclosed in “Doséclair”). For a “unitary” beam, the step corresponds to the estimation of the gradient of the dose, which estimation (or calculation) is in its details obtained by means of an analytic function for this gradient of dose (deposited). To obtain this analytic function, there is undertaken the calculation of the gradient of the composition of a projection function with a function of dose deposited in a homogeneous medium (in a voxel); this analytic function for calculating dose gradient being calculated gradually in the mesh cells traversed by the beam. There may be a plurality of ionizing beams, which are either independent or at least partially dependent (through their parameters). The optimization of the treatment plan can be performed on the basis of calculations. By means of a cost function, the irradiation of the tissues can be optimized, thus corresponding to the sought-after objective (maximization of the irradiation of the tumor while minimizing the effects on the neighboring tissues). The method can comprise the displaying of the treatment plan (for example optimized) and/or of numerical values associated with the geometric parameters and with the fluence of one or more beams. The results and various numerical values (including guiding options for the heads, or even simulations of actions, etc) can be displayed. On account of the construction of the procedure, sufficiently fast temporal repetitions allow irradiations via heads that are moving (rather than static) around the patient.

In a development, a computer program product comprising code instructions makes it possible to perform any one of the steps of the method when the program is executed on a computer. The present invention can be implemented on the basis of hardware elements (system 490). Any one of the steps of the method can be implemented on computer, which generally comprises calculation means 491, random-access or non-permanent memory 492, input and output or I/O or network means 493 (wired and/or wireless network connectivity or bus or peripherals for audio-visual input or restitution) as well as storage means 494 (for example mass storage). The computer program product can be encoded on a computer readable medium. The medium can be electronic, magnetic, optical, or electromagnetic (non-exhaustive). An implementation of the method can be local and/or via remote access. For example, at least part of the calculation and/or storage means can be accessible by means hosted in the network (“Cloud Computing”) modulo the requirements in regard to latency time, which may themselves be optimized. The traffic can be encrypted. The viewing means 493 may in particular comprise one or more projectors (including laser) to guide the orientation of the sensors and/or their movements, virtual and/or augmented reality headsets, haptic feedbacks (force feedback, touch surface, etc).

In a development, an ionizing particle is a photon and/or an electron and/or a hadron and/or a proton. Stated otherwise, the method according to the invention can be applied to procedures for hadrontherapy, protontherapy and electron radiotherapy.

### ANNEX

(1) Reference Frame [symbol] _{F}
Right-handed orthonormal reference frame (O; x; y; z) whose origin O is the center of the phantom. Termed “phantom” reference frame.
(2) Reference Frame [symbol] _{f}
Right-handed orthonormal reference frame (M₀^{f}; e₁; e₂; e₃) called [symbol] _{f}, is associated with an elementary beam. The origin M₀^{f} is the center of the origin of the beam. Termed “beam” reference frame.
(3) Tait-Bryan z-y-x Variant of the Euler Angles
This variant makes it easier to write expressions for the optimization of the orientation of the beam. It is defined by a rotation by α about the axis z, a rotation by β about the new axis y, rotation by γ about the new axis x. These three rotations suffice to define on the basis of the triplet of orthonormal vectors of the “phantom” reference frame the triplet of orthonormal vectors of the “beam” reference frame [symbol] _{f} as:

$$[ {e}_{1} ; {e}_{2} ; {e}_{3} ] = [ \begin{matrix}cos β cos α & \begin{matrix}cos α sin γ sin β - \\ cos γ sin α\end{matrix} & \begin{matrix}cos γ cos α sin β + \\ sin α sin γ\end{matrix} \\ cos β sin α & \begin{matrix}cos α cos γ + \\ sin α sin β sin γ\end{matrix} & \begin{matrix}- cos α sin γ + \\ cos γ sin β sin α\end{matrix} \\ - sin β & cos β sin γ & cos γ cos β\end{matrix} ]$$

The angles are bounded: αε]−π, π], βε]−π, π], γε]−π, π]

### (4) Projection

Tensor [symbol] of order 2, matrix of dimension 4×4, for switching from [symbol] _{F} to [symbol] _{f}

$$= [ \begin{matrix}{f}_{11} & {f}_{12} & {f}_{13} & {f}_{14} \\ {f}_{21} & {f}_{22} & {f}_{23} & {f}_{24} \\ {f}_{31} & {f}_{32} & {f}_{33} & {f}_{34} \\ 0 & 0 & 0 & 1\end{matrix} ]$$

(5) Tensor for Switching from [symbol] _{F} to [symbol] _{f}, Denoted [symbol] .

$$\begin{matrix}= ⊗ \\ = [ \begin{matrix}cos β cos α & \begin{matrix}cos α sin γ sin β - \\ cos γ sin α\end{matrix} & \begin{matrix}cos γ cos α sin β + \\ sin α sin γ\end{matrix} & - 〈 {e}_{1} , \overline{{OM}_{0}^{f}} 〉 \\ cos β sin α & \begin{matrix}cos α cos γ + \\ sin α sin β sin γ\end{matrix} & \begin{matrix}- cos α sin γ + \\ cos γ sin β sin α\end{matrix} & - 〈 {e}_{2} , \overline{{OM}_{0}^{f}} 〉 \\ - sin β & cos β sin γ & cos γ cos β & {}_{34} - 〈 {e}_{3} , \overline{{OM}_{0}^{f}} 〉 \\ 0 & 0 & 0 & 1\end{matrix} ]\end{matrix}$$

The coefficient [symbol] ₃₄ conveys the heterogeneities of the medium, including the “void” region between the beam origin and the patient's body. This coefficient is calculated by the axial propagation mechanism described in “Doséclair”.

### (6) Lineal Attenuation Coefficient μ.

The fluence at the depth z in a homogeneous medium with lineal attenuation coefficient μ is given by: φ(z)=φ(0). e^{-μz}.

### (7) Recursive Formula for the Component

$${}_{34}^{〈 s + 1 〉} = \frac{{μ}^{〈 s 〉}}{{μ}^{〈 s + 1 〉}} {}_{34}^{〈 s 〉} + ( \frac{{μ}^{〈 s 〉}}{{μ}^{〈 s + 1 〉}} - 1 ) 〈 {e}_{3} , \overline{{OM}_{0}^{f}} 〉 + ( \frac{{μ}^{〈 s 〉}}{{μ}^{〈 s + 1 〉}} - 1 ) {λ}^{〈 s + 1 〉}$$

Where λ^{(s+1)} is the distance traveled from the origin point M₀^{f} of the beam in the direction e₃ in order to reach the (geometric) depth of the entry barycenter of the following SBIM.

### (8) Total Dose

At any point P:

$$Dose ( P ) = φ ( 0 ) {\sum }_{s} {k}_{〈 s 〉} ( P ) \times {Dose}_{〈 s 〉} ( P )$$

Where φ(0) is the fluence of the beam on entry to the meshed phantom.
The weighting [symbol] (P) depends on the position and shape of the SBIM. The total dose Dose(P) is a mixture of the various Dose₍ₛ₎(P).

$${\sum }_{s} {k}_{〈 s 〉} ( P ) = 1$$

### (9) Spherical Coordinates

M₀^{f} can then be characterized by its spherical coordinates: the angles θ_{f} (longitude) and φ_{f} (colatitude).

$${M}_{0}^{f} = R ( \begin{matrix}sin ϕ cos θ \\ sin ϕ sin θ \\ cos ϕ\end{matrix} )$$

Ω_{f}=(Ψ_{f}_{¹}φ_{f}) is the vector of the parameters of the elementary beam to be optimized
Ψ_{f} is the vector of the ballistic (or geometric) parameters, that is to say of the five angles (θ_{f}_{¹}φ_{f}_{¹}α_{f}_{¹}β_{f}_{¹}γ_{f}_{¹}).
φ_{f} is the fluence of the beam.

### (10) Dose Calculation

Dose(P)=φ_{f}·Dose^{f}(P)=φ_{f}·Σₛ=_{{SBIMs of the beam}}k₍ₛ₎(P)×Dose₍ₛ₎(P)Dose₍ₛ₎(P)=H_{[mat]}(proj₍ₛ₎(P))

### (11) Dose Gradient Calculation

Chain rule of differentiation:

$$\frac{\partial {Dose}_{〈 s 〉} ( P )}{\partial {ψ}_{f}} = \frac{\partial {H}_{〚 mat 〛} ( {proj}_{〈 s 〉} ( P ) )}{\partial {proj}_{〈 s 〉} ( P )} \times \frac{\partial {proj}_{〈 s 〉} ( P )}{\partial {ψ}_{f}} ∀ {ψ}_{f} ∈ {Ψ}_{f}$$

### (12) Case of a Spline Distribution Function

Example of functions for the distribution in a homogeneous medium H_{[mat]} (splines of order 3): ax²+by²+cx²y+dxy²+ex+fγ+gz, where x, y and z are the coordinates of the projected point.

$$gradient \frac{\partial {H}_{mat} ( P )}{\partial {P}^{〈 s 〉} ( P )} \text{:}$$

$$\frac{\partial {H}_{〚 mat 〛} ( {proj}_{〈 s 〉} ( P ) )}{\partial {proj}_{〈 s 〉} ( P )} = [ \begin{matrix}3 α {x}^{2} + 2 cxy + {dy}^{2} + e \\ 3 {by}^{2} + {cx}^{2} + 2 dxy + f \\ g\end{matrix} ]$$

### (13) Gradient of the Tensor

Parameters of unitary elementary beam Ψ_{f}=(θ_{f}_{¹}φ_{f}_{¹}α_{f}_{¹}β_{f}_{¹}γ_{f})
Gradient of the tensor with respect to α_{f}, denoted G_{α}, is called a gradient tensor α.
The formulae derivable between the coefficients of the tensor and the parameters make it possible to calculate the gradient at any point, with the exception of the coefficient t₃₄ which can be calculated by propagation (gradual calculation).

### (14) Case of IMRT Beams (“Step and Shoot” Treatment)

IMRT beams: the elementary beams of this IMRT are oriented by the same triplet (e₁, e2, e3), and therefore all share the same parameter triplet (αᵣ; βᵣ; γᵣ).
The indices of p (respectively q) lie between 1 and Np (respectively Nq). Conventionally in a clinical setting, Np=Nq=10.
The origin-center of the elementary beam (p, q) is defined by:

$$\vec{{OM}_{0}^{r}} + ( p - ( Np + 1 ) / 2 ) δ \vec{{e}_{1}} + ( q - ( Nq + 1 ) / 2 ) δ \vec{{e}_{2}} , \\ \frac{\partial Dose ( P )}{\partial {ψ}_{r}} = {\sum }_{p = 1}^{{N}_{p}} {\sum }_{q = 1}^{{N}_{q}} {w}_{( p , q )}^{r} \frac{\partial {Dose}^{r , ( p , q )} ( P )}{\partial {ψ}_{r}}$$

The gradient of the dose with respect to the fluence matrix can be calculated: it suffices to know the dose deposited by each elementary beam.

### (15) Cost Function

$${E}_{min} ( P ) \{ \begin{matrix}= {( {D}_{ref} ( P ) - D ( P ) )}^{2} & {ifD}_{ref} ( P ) > D ( P ) \\ 0 & {ifD}_{ref} ( P ) \leq D ( P )\end{matrix}$$

where Eₘᵢₙ(P) is the value of the cost function at the position P, D_{ref} (P) is the reference dose at the position P, D(P) is the dose deposited by the current protocol at the position P.

### (16) Gradient Descent

With an objective function G, for example which measures the disparity between the dose deposited and the dose prescribed:

minG(x) [symbol] ∇G(x)=0

The variable x consists of the set of geometric parameters of the beams and their associated intensities.
The search for x is made iteratively, according to the general relation for the gradients:

xₖ₊₁=xₖ+a·dₖ

where k designates the index number of the iteration, a shows the size of the search step (variable or invariable) and dₖ is the direction of descent.
Steepest descent procedure

dₖ=−∇G(xₖ) hence: xₖ₊ᵢ=xₖ−a·∇G(xₖ); with fixed or dynamic step size

1. A method for estimating a dose gradient with respect to the parameters of a beam of ionizing particles, the dose being deposited by said beam in a voxel of a phantom of a patient, said phantom being meshed, each mesh cell of the phantom comprising voxels of one and the same material, the parameters of the beam comprising a fluence parameter and geometric parameters, said method comprising the
determination of the analytic function for the gradient of the dose, deposited per mesh cell, with respect to the parameters of the beam; and the
determination of the estimation of the gradient of the dose in the voxel.

2. The method for estimating a dose gradient as claimed in claim 1, the estimation of the dose gradient being the gradient of the dose estimation, the estimation of the dose deposited in the voxel comprising the
determination of an analytic function for calculating dose deposited per mesh cell, said function being obtained by propagating the parameters of analytic functions of dose deposited gradually in the neighboring mesh cells traversed by the beam; and the
determination of the estimation of the dose deposited in the voxel.

3. The method as claimed in claim 1, comprising a plurality of independent beams and furthermore comprising the determination of the dose deposited in the voxel by summing the doses deposited by each independent beam.

4. The method as claimed in claim 1, comprising a plurality of independent beams and furthermore comprising the estimation of the gradient of dose deposited in the voxel by summing the gradients of doses deposited by each independent beam.

5. The method as claimed in claim 1, comprising a plurality of at least partially dependent beams, some of their geometric parameters being interdependent, and furthermore comprising the determination of the dose deposited in the voxel.

6. The method as claimed in claim 2, the determination of an analytic function for calculating gradient of dose deposited in an arbitrary voxel of the phantom being obtained through the gradient of the composition of the projection function with the gradient of the dose deposited in a voxel; the determination of the analytic function for calculating dose gradient being calculated gradually in the mesh cells traversed by the beam.

7. The method as claimed in claim 1, the geometric parameters of a beam comprising five degrees of freedom, corresponding to the information regarding the origin, rotation and direction of the beam in space.

8. The method as claimed in claim 1, further comprising:
the selection of a differentiable cost function;
the determination of the gradient of said cost function with respect to the parameters of the beam, said gradient being obtained by composition of the derivative of the cost function with respect to the dose with the gradient of the dose with respect to the parameters; and
the minimization of said cost function corresponding to the obtaining of a local minimum of the cost function.

9. The method as claimed in claim 8, comprising the determination and the minimization of the cost function for a plurality of voxels which may or may not belong to one and the same mesh cell.

10. The method as claimed in claim 1, for which an ionizing particle is a photon and/or an electron and/or a hadron and/or a proton.

11. The method as claimed in claim 1, further comprising the displaying of the treatment plan and/or of numerical values associated with the geometric parameters and with the fluence of a beam.

12. The method as claimed in claim 1, a beam moving along a trajectory comprising at least two control points and intermediate trajectory points.

13. The system comprising means for implementing the steps of the method as claimed in claim 1, comprising a servo-controlled robotic arm carrying the beam or beams or an arc therapy system or a system combining movements of translation of the patient and of rotation of equipment carrying the beam or beams.

14. The system as claimed in claim 13, the irradiation parameters associated with the intermediate trajectory points being deduced by interpolation of the irradiation parameters associated with the control points.

15. A computer program product, said computer program comprising code instructions making it possible to perform the steps of the method as claimed in claim 1, when said program is executed on a computer.
